(19) United States
(12) Patent Application Publication
(10) Pub. No.: US 2025/0130165 A1
(43) Pub. Date: Apr. 24, 2025

(54) NEAR-INFRARED-SPECTRUM-BASED APPARATUS FOR ON-SITE RAPID MEASUREMENT OF NITROGEN AND PHOSPHORUS CONTENT IN LIQUID MANURE

(71) Applicant: Agro-Environmental Protection Institute, Ministry Of Agriculture And Rural Affairs, Tianjin (CN)

(72) Inventors: Run ZHAO, Tianjin (CN); Suli ZHI, Tianjin (CN); Zengjun YANG, Tianjin (CN); Keqiang ZHANG, Tianjin (CN)

(21) Appl. No.: 18/990,603

(22) Filed: Dec. 20, 2024

(30) Foreign Application Priority Data
Aug. 9, 2022 (CN) 2022109465225

(51) Int. Cl. G01N 21/3577; G01N 21/25; G01N 21/359; G01N 33/487

(52) U.S. Cl. CPC G01N 21/3577; G01N 21/255; G01N 21/359; G01N 33/4875; G01N 2201/08

(57) ABSTRACT

An on-site detection device for nitrogen and phosphorus contents in liquid feces based on near infrared spectroscopy, including a box body, a box plate and a box cover, an independent detection part is formed, in which a detecting component and a sensor group are arranged; the detecting component, a temperature regulating component, a gas circulation component and the sensor group are all electrically connected to the controlling component, thus to achieve precise on-site detection. This disclosure utilizes a detection component to collect sample spectral information with 360°, minimizing the impact of temperature, humidity, and interfering gases in the detection environment with the assistance of temperature regulating and gas circulation components. This ensures accurate and stable spectral signal detection, thereby addressing the technical bottlenecks and weak stability in conventional laboratory chemical determinations. It also resolves the challenges posed by the treatment environment of feces matter during on-site detection.

### CROSS REFERENCE TO RELATED APPLICATIONS

This application is a continuation application of international application number PCT/CN2023/111386, filed Aug. 7, 2023, which claims priority to Chinese patent application 202210946522. 5, filed on Aug. 9, 2022. The contents of these applications are incorporated herein by reference in their entirety.

### TECHNICAL FIELD

The present disclosure relates to the technical field of detecting nitrogen and phosphorus contents in liquid feces, and relates to an on-site rapid determination device for total nitrogen and total phosphorus contents in liquid feces in an intensive dairy farm, in particular to an on-site detection device for nitrogen and phosphorus contents in liquid feces based on near infrared spectroscopy.

### BACKGROUND

Nitrogen and phosphorus are important considerations for the scientific return of manure to the fields in large-scale dairy farms. The manure transportation and treatment process of intensive dairy farms in China is complex and changeable, with a long process and repeated cycles. Therefore, traditional laboratory chemical detection has technical bottlenecks such as poor timeliness, low accuracy, and weak stability. Rapid and accurate detection of nitrogen and phosphorus contents in liquid feces is a pressing challenge for dairy farms striving for integrated crop-livestock systems. However, the difficulties of on-site rapid detection include lack of portable detection device and environment influence that bring by on-site detection. Specifically, since the on-site detection is a manure treatment facility such as aeration tanks and oxidation ponds, some specific environments are relatively humid, and the air flow is also filled with gases such as ammonia and hydrogen sulfide. The presence of the above gases greatly affects the results of on-site detection of nitrogen and phosphorus contents in liquid feces. Therefore, there is an urgent need for an on-site rapid detection device for nitrogen and phosphorus contents in liquid feces based on near-infrared spectroscopy that can perform liquid feces detection on-site at dairy farms with high efficiency and accuracy, and provide technical support for the scientific return of liquid feces to the fields.

No patent literature highly relevant to this application were found through the search.

### SUMMARY

The purpose of the present disclosure is to overcome the shortcomings of the prior arts and provide an on-site detection device for nitrogen and phosphorus contents in liquid feces based on near infrared spectroscopy, the on-site detection device has a novel structure and is easy to carry, and can achieve efficient and accurate detection results. The on-site detection device solves the technical bottlenecks of poor timeliness, low accuracy, and weak stability of conventional laboratory chemical detection, and also solves the problem that on-site detection is easily affected by the liquid feces environment.

To realize the above objectives, the present disclosure provides an on-site detection device for nitrogen and phosphorus contents in liquid feces based on near infrared spectroscopy, including: a box body, a box plate and a box cover, an upper end of the box body is defined with a box opening, the box plate is installed at the box opening, and the box cover is hinged on one side of the box body; wherein, one side of the box body is enclosed by a surrounding plate to form an independent detection part, in which a detecting component and a sensor group are arranged; and another side of the box body is a controlling part, in which a controlling component is arranged; a temperature regulating component and a gas circulation component are also arranged inside the box body; and the detecting component, the temperature regulating component, the gas circulation component and the sensor group are all electrically connected to the controlling component; and the controlling component in configured to receive an infrared spectrum signal fed back by the detecting component, and analyzes the nitrogen and phosphorus contents of the to be detected liquid feces sample through the characteristics of the infrared spectrum signal; the controlling component is configured to receive a signal value transmitted from the sensor group, and starts the temperature regulating component and the gas circulation component according to the signal value, and the controlling component is also configured to keep the detection environment of the detection part stable to maintain a constant temperature and interference-free detection environment for the detecting component.

Furthermore, the detecting component includes a spectrum collection mechanism and a hollow rotating mechanism, the hollow rotating mechanism is arranged on an upper part of a sample port of an integrating sphere of the spectrum collection mechanism, the hollow rotating mechanism is coaxially embedded with a sample cup embedding frame, a sample cup is coaxially installed in the sample cup embedding frame; and an infrared temperature sensor is installed on the sample cup embedding frame to monitor a detection temperature of the to be detected liquid feces sample in the sample cup in real time, and a vibration sensor is installed on a mounting frame of the hollow rotating mechanism or on an outside of the integrating sphere to monitor in real time and reduce an influence caused by vibrating of the to be detected liquid feces sample on the detection result.

Furthermore, the spectrum acquisition mechanism includes a spectrometer and the integrating sphere, the integrating sphere is installed on one side of the detection part, the spectrometer is installed on another side of the detection part, and the integrating sphere is communicated with the spectrometer through an optical fiber; the sample port is formed on a top of the integrating sphere; the hollow rotating mechanism includes the mounting frame, the mounting frame is mounted on a lower surface of the box plate, and a lower end of the mounting frame is coaxially inserted with the integrating sphere, a hollow electric rotating platform is installed in the mounting frame, a middle part of the hollow electric rotating platform is a hollow part, and the sample cup embedding frame is coaxially installed in the hollow part, and the box plate corresponding to the position of the sample cup embedding frame is defined with a through hole, and an upper part of the sample cup embedding frame passes through the through hole.

Furthermore, the temperature regulating component includes a CPU (central processing unit) processor wrapping box, an electronic refrigeration sheet and a gas circulation pump; wherein the CPU processor wrapping box is configured to collect heat emitted by a CPU processor, the CPU processor wrapping box is provided with a box air outlet; and a pump air inlet and a pump air outlet of the gas circulation pump are both provided with a three-way valve; and the pump air inlet of the gas circulation pump includes a first pump air inlet and a second pump air inlet, the pump air outlet of the gas circulation pump includes a first pump air outlet and a second pump air outlet; wherein the box air outlet of the CPU processor wrapping box is connected to the first pump air inlet of the gas circulation pump through a first pipeline, and the first pump air outlet of the gas circulation pump is connected to the detection part through a second pipeline; and the electronic refrigeration sheet is arranged on a surrounding plate close to one side of the controlling part, a side A of the electronic refrigeration sheet is a hot side located in the controlling part, and a side B of the electronic refrigeration sheet is a cold side located in the detection part; and the gas circulation component comprises a three-effect air filter, the gas circulation pump, a first circulation pipeline and a second circulation pipeline; wherein the gas circulation component and the temperature regulating component share the gas circulation pump; and a plate air outlet is defined on a corner of the surrounding plate, the plate air outlet is connected to the second pump air inlet of the gas circulation pump through the first pipeline, and the second pump air outlet of the gas circulation pump is connected to an air inlet of the triple-effect air filter through the second circulation pipeline, and the air outlet of the triple-effect air filter is connected to the detection part.

Furthermore, the sensor group includes an ammonia concentration sensor, a temperature sensor and a humidity sensor, and the temperature sensor is configured to monitor a temperature in the detection part and transmit a temperature signal to the controlling component; and the humidity sensor is configured to monitor humidity in the detection part and transmit a humidity signal to the controlling component; and the ammonia concentration sensor is configured to monitor a ammonia content in the detection part and transmit an ammonia content signal to the controlling component.

Furthermore, the triple-effect air filter includes a hollow glass cylindrical shell with through holes at both ends, rubber sealing plugs are provided at both ends of the hollow glass cylindrical shell, a card slot is defined on the box plate, and circular holes are defined on both sides of the card slot; and a slot cover is hinged on the card slot; wherein the circular holes on a same side is coaxially arranged at an end of an air inlet pipe, and the annular sealing plugs are embedded in the circular holes.

Furthermore, the hollow glass cylindrical shell is filled with acidic gas adsorption particles, neutral gas adsorption particles and alkaline gas adsorption particles in sequence.

Furthermore, the controlling component includes a CPU processor, a graphics card and a power supply; a display is also provided in the box cover, and a keyboard, a USB (universal serial bus) interface and an exhaust fan are also provided in the controlling part; the graphics card, the power supply, the display, the keyboard, the USB interface and the exhaust fan are all electrically connected to the processor, and the keyboard and the USB interface are arranged through the box plate, the exhaust fan dissipates heat for the graphics card and the power supply, and a plurality of exhaust holes are arranged on the box plate corresponding to the exhaust fan; the CPU processor receives the data transmitted by each elements of the detecting component for processing to obtain a rapid and accurate determination of the total nitrogen and total phosphorus content in the unknown liquid feces sample, and monitors the detection situation in real time through the display.

Furthermore, the mounting frame includes a limiting slot and a U-shaped slot, the limiting slot is a hollow rectangular structure that passes through from a top to a bottom; wherein a top of the limiting slot is coaxially fixed with the U-shaped slot, and a bottom of the U-shaped slot is bolted to install the hollow electric rotating platform to achieve the coaxial arrangement of the hollow part of the hollow electric rotating platform and the sample port at the top of the integrating sphere; wherein tops of groove walls on both sides of the U-shaped groove are vertically formed with wing plates outside the groove, the wing plates are flush with the top of the surrounding plate, and reinforcing ribs are also provided on an outer side of the groove wall of the U-shaped groove.

Furthermore, the sample cup embedding frame includes an insertion tube and a limit frame; wherein the insertion tube is a hollow tube that passes through from a top to a bottom, the insertion tube is adapted to the hollow part of the hollow electric rotating platform; and the top of the insertion tube is coaxially fixed with the limit frame, the limit frame is formed by two layers of hollow discs coaxially fixed by connecting columns; and the hollow part of the hollow disc is a hollow portion, the hollow portion of the hollow part and the hollow part of the insertion tube are coaxial to form a sample cup embedding groove, and the limit frame is located on an upper surface of the box plate.

The advantages and positive effects of the present disclosure are as follow.

The present disclosure adopts a design concept of the integrated portable device, which realizes the functions of light source generation, spectrum collection, sample rotating in a fixed angle, collector constant temperature control, computer data analysis, monitor on-site display, real-time data uploading, etc., meeting the needs of on-site rapid detection.

The spectrum collection mechanism, hollow rotating mechanism and temperature regulating component of the present disclosure. The hollow rotating mechanism can realize 360 degree blind-angle-free auxiliary spectrum collection mechanism to collect sample spectrum information, and realize the maximum collection of dairy cow dung sample information per unit time. The temperature regulating component can adjust the temperature of the detection environment when collecting sample spectrum information, so that the detection part is an independent constant temperature room, and the detection element is maintained at a constant temperature to detect the sample, effectively reducing the error caused by the detection. The gas circulation component can perform real-time dehumidification and interference gas removal operations on the detection environment in the detection part, providing a non-interference detection environment for the detecting component, ensuring the accuracy of spectral signal detection.

Comparing with the existing laboratory detection, the present disclosure does not require pretreatment such as acid addition, titration, dilution, filtration, etc., in the whole process. It meets the urgent need for on-site rapid detection and analysis of nutrients in dairy manure returned to the field. It solves the technical bottlenecks of poor timeliness, low accuracy, and weak stability of conventional laboratory chemical determinations, and also solves the problem that on-site detection is easily affected by the manure treatment environment.

### BRIEF DESCRIPTION OF THE DRAWINGS

FIG. 1 is a schematic structural diagram of an on-site detection device for nitrogen and phosphorus contents in liquid feces based on near infrared spectroscopy according to an embodiment of the present disclosure.

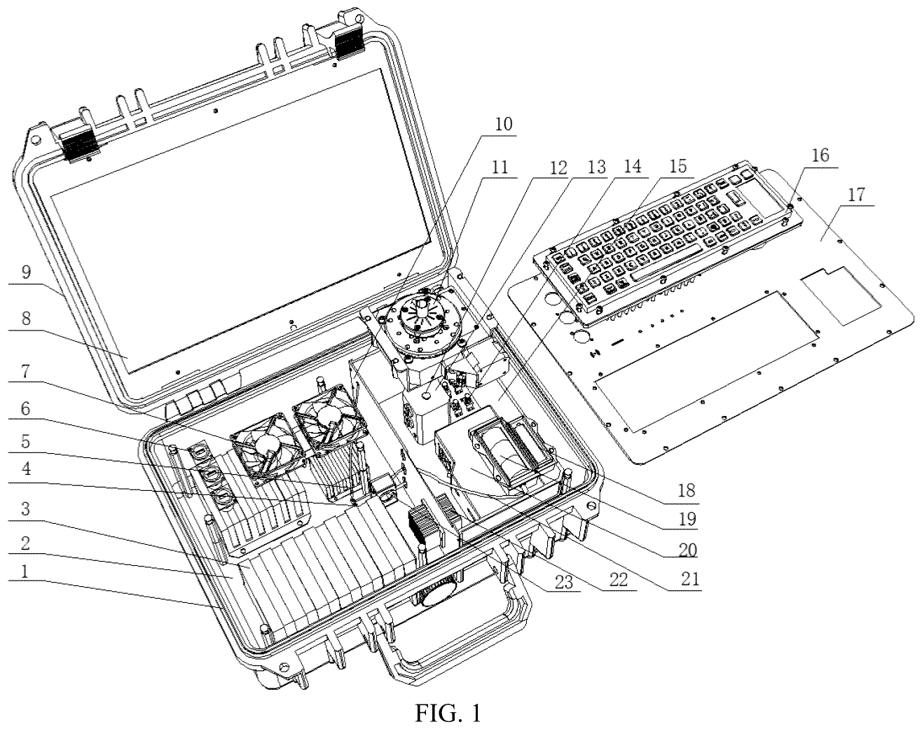

FIG. 2 is a perspective view of the an on-site detection device for nitrogen and phosphorus contents in liquid feces based on near infrared spectroscopy of FIG. 1.

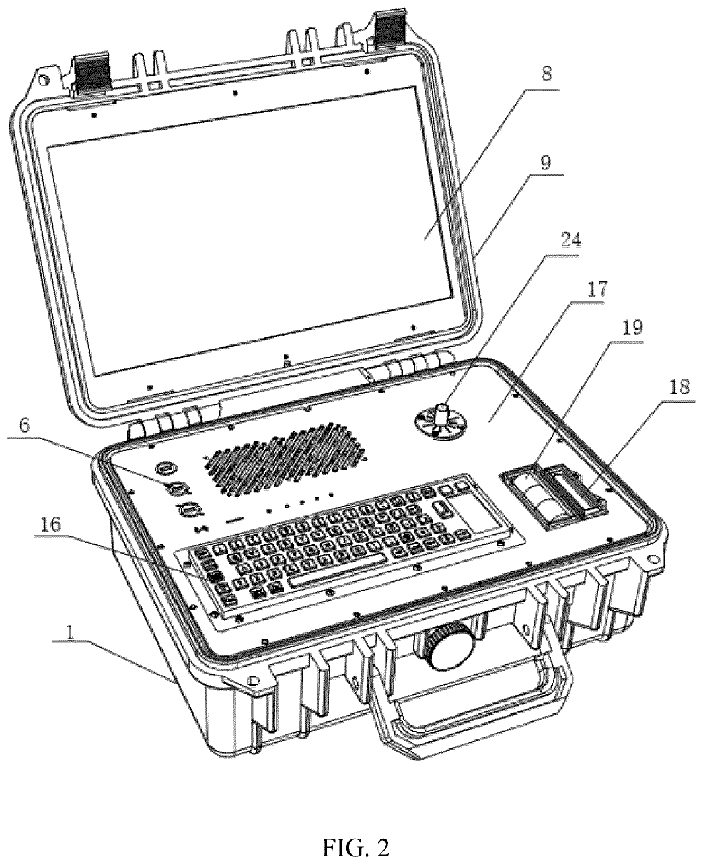

FIG. 3 is a schematic structural diagram of a temperature regulating component and a gas circulation component of the on-site detection device of FIG. 1.

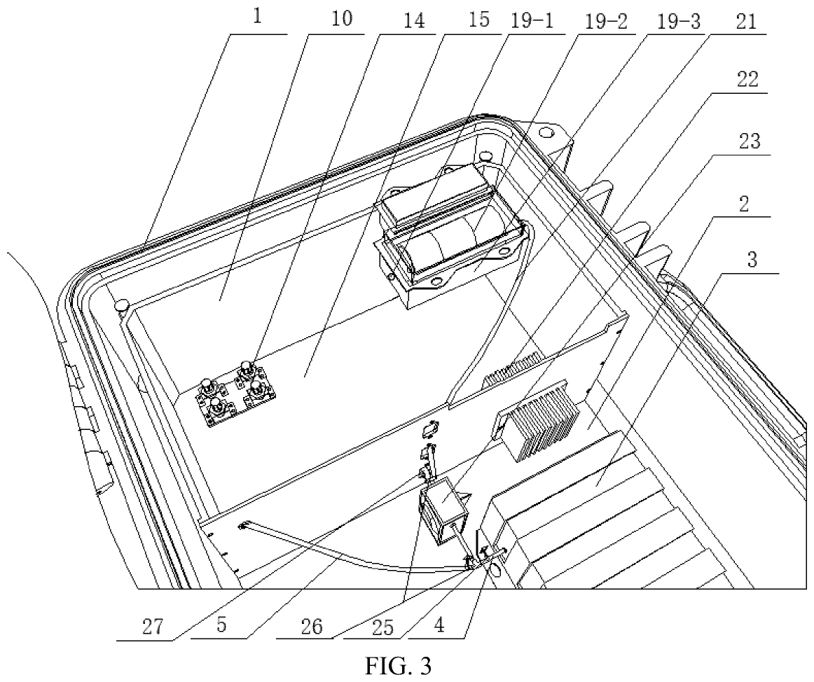

FIG. 4 is a schematic structural diagram of a detecting component of the on-site detection device of FIG. 1.

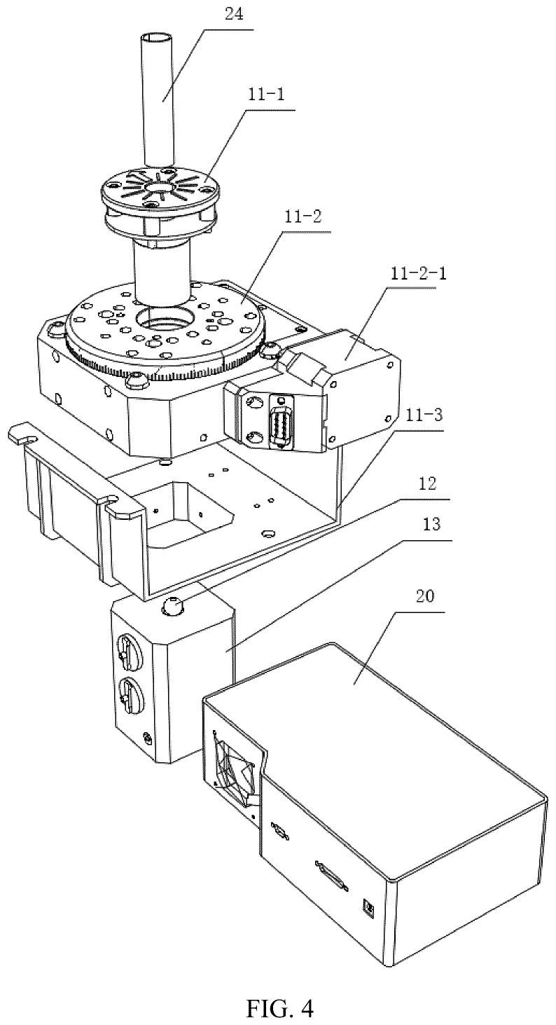

### LABELS AND DESCRIPTIONS

1 box body, 2 controlling part, 3 CPU processor wrapping box, 4 first pipeline, 5 first circulation pipeline, 6 USB interface, 7 exhaust fan, 8 display, 9 box cover, 10 surrounding plate, 11 hollow rotating mechanism, 12 sample port, 13 integrating sphere, 14 sensor group, 15 detection part, 16 keyboard, 17 box plate, 18 sample cup temporary storage box, 19 three-effect air filter, 20 spectrometer, 21 second circulation pipeline, 22 electronic refrigeration sheet, 23 gas circulation pump, 24 sample cup, 25 solenoid valve, 26 three-way valve, 27 second pipeline, 11-1 sample cup mounting rack, 11-2 hollow electric rotating platform, 11-3 mounting frame, 11-2-1 servo stepping motor, 19-1 circular hole, 19-2 hollow glass cylindrical shell, 19-2 card slot.

### DETAILED DESCRIPTION OF THE EMBODIMENTS

The technical solutions in the embodiments of the present disclosure will be described clearly and completely with reference to the accompanying drawings in the embodiments of the present disclosure. Obviously, the described embodiments are only a part of the embodiments of the present disclosure rather than all of them. Based on the embodiments in the present disclosure, all other embodiments obtained by those skilled in the art without creative work shall fall within the scope of protection of the present disclosure.

The embodiment provides an on-site detection device for nitrogen and phosphorus contents in liquid feces based on near infrared spectroscopy, including a box body 1, a box plate 17 and a box cover 9. An upper end of the box body 1 is defined with a box opening, the box plate 17 is installed at the box opening. The box cover 9 is hinged on one side of the box body. One side of the box body is enclosed by a surrounding plate 10 to form an independent detection part 15, in which a detecting component and a sensor group 14 are arranged. The other side of the box body 1 is a controlling part 2, in which a controlling component is arranged. A temperature regulating component and a gas circulation component are also arranged inside the box body. The detecting component, the temperature regulating component, the gas circulation component and the sensor group are all electrically connected to the controlling component. The controlling component in configured to receive an infrared spectrum signal fed back by the detecting component, and analyzes the nitrogen and phosphorus contents of the to be detected liquid feces sample through the characteristics of the infrared spectrum signal. The controlling component is configured to receive a signal value transmitted from the sensor group, and starts the temperature regulating component and the gas circulation component according to the signal value, and the controlling component is also configured to keep the detection environment of the detection part 2 stable to maintain a constant temperature and interference-free detection environment for the detecting component. The relevant infrared spectral signal recognition software are adopted to analysis spectral signals. All controls are implemented via CPU, and the methods and components are based on existing technology. This embodiment does not impose any special limitations; its embodiment revolves around overcoming the interference caused by the detection environment on the detection results in the existing technology.

The component includes a spectrum collection mechanism and a hollow rotating mechanism 11, the hollow rotating mechanism 11 is arranged on an upper part of a sample port 12 of an integrating sphere 13 of the spectrum collection mechanism. The hollow rotating mechanism is 11 coaxially embedded with a sample cup embedding frame 11-1, a sample cup 24 is coaxially installed in the sample cup embedding frame 11-1. And an infrared temperature sensor is installed on the sample cup embedding frame 11-1 to monitor a detection temperature of the to be detected liquid feces sample in the sample cup in real time, to reduce influence caused by temperature difference of the to be detected liquid feces samples. A vibration sensor is installed on a mounting frame 11-3 of the hollow rotating mechanism 11 or on an outside of the integrating sphere 13 to monitor in real time and reduce an influence caused by vibrating of the to be detected liquid feces sample on the detection result.

The spectrum acquisition mechanism includes a spectrometer 20 and the integrating sphere 13. The integrating sphere 13 is installed on one side of the detection part 15, the spectrometer 20 is installed on the other side of the detection part 15, and the integrating sphere 13 is connected to the spectrometer 20 through an optical fiber. The sample port 12 is formed on a top of the integrating sphere 13. The hollow rotating mechanism 11 includes the mounting frame 11-3, the mounting frame 11-3 is mounted on a lower surface of the box plate 17. A lower end of the mounting frame 11-3 is coaxially inserted with the integrating sphere 13. A hollow electric rotating platform 11-2 is installed in the mounting frame 11-3, a middle part of the hollow electric rotating platform 11-2 is a hollow part. And the sample cup embedding frame 11-1 is coaxially installed in the hollow part. The sample cup embedding frame 11-1 is coaxially installed with the sample cup 24. The sample cup 24 is arranged coaxially with the sample port 12 at the top of the integrating sphere 13. A through hole is defined in the box plate 17 corresponding to the position of the sample cup embedding frame 11-1, which facilitates the passage through the upper part of the sample cup embedding frame 11-1, allowing for easy retrieval and replacement of the sample cup within the sample cup embedding frame 11-1. The hollow electric rotating platform 11-2 drives the sample cup 24 mounted within the sample cup embedding frame 11-1 to rotate uniformly at a fixed angle, enabling the spectral acquisition mechanism to collect spectral information from the same to be detected liquid feces sample at multiple angles, thereby avoiding the impact of sample inhomogeneity on the detection results.

The temperature regulating component includes a CPU (central processing unit) processor wrapping box 3, an electronic refrigeration sheet 22 and a gas circulation pump 23. The CPU processor wrapping box is configured to collect heat emitted by a CPU processor. The CPU processor wrapping box 3 is provided with a box air outlet. A pump air inlet and a pump air outlet of the gas circulation pump are both provided with a three-way valve 26. The three-way valve 26 also known as three-way pipe, an electromagnetic valve 25 is installed on each pipeline. The pump air inlet of the gas circulation pump includes a first pump air inlet and a second pump air inlet. And the pump air outlet of the gas circulation pump includes a first pump air outlet and a second pump air outlet. The box air outlet of the CPU processor wrapping box 3 is connected to the first pump air inlet of the gas circulation pump through a first pipeline, and the first pump air outlet of the gas circulation pump is connected to the detection part through a second pipeline 27. The electronic refrigeration sheet is arranged on a surrounding plate 10 close to one side of the controlling part 2, a side A of the electronic refrigeration sheet is a hot side located in the controlling part, and a side B of the electronic refrigeration sheet is a cold side located in the detection part. Heat generated by the CPU processor is used to warm the detection part. When the temperature is below the detection temperature of 20-25° C., the gas circulation pump is activated to increase the temperature. When the temperature exceeds the detection temperature of 20-25° C., the gas circulation pump is turned off, and the electronic cooling plate is activated to lower the temperature within the detection part.

The gas circulation component includes a three-effect air filter 19, the gas circulation pump 23, a first circulation pipeline 5 and a second circulation pipeline 21. The gas circulation component and the temperature regulating component share the gas circulation pump. To regulate the gas environment in the detection part and reduce the impact of ammonia nitrogen in the ambient air on infrared light absorption, a gas circulation component is installed in the detecting component. The power source is supplied by the gas circulation pump 23 in the temperature regulating component. A plate air outlet is defined on a corner of the surrounding plate 10, the plate air outlet is connected to the second pump air inlet of the gas circulation pump through the first pipeline, and the second pump air outlet 21 of the gas circulation pump is connected to an air inlet of the triple-effect air filter 19 through the second circulation pipeline 21, and the air outlet of the triple-effect air filter is connected to the detection part. When the sensor group 14 detects ammonia concentration and humidity values exceeding the standard range (normal operating relative humidity standard: below 50% RH; NH3 concentration standard: below 0.2 ppm), the gas circulation pump and the gas circulation component corresponding to the second air inlet and second air outlet of the gas circulation component are activated to purify the environment within the detection part until it meets the required standards.

The sensor group 14 includes an ammonia concentration sensor, a temperature sensor and a humidity sensor. The temperature sensor is configured to monitor a temperature in the detection part and transmit a temperature signal to the controlling component; and the humidity sensor is configured to monitor humidity in the detection part and transmit a humidity signal to the controlling component. To avoid the impact of various detection elements in the detection component on the results at different temperatures and to reduce the difficulty of temperature compensation. The ammonia concentration sensor is configured to monitor a ammonia content in the detection part and transmit an ammonia content signal to the controlling component. While the three-effect air filter is used to absorb interfering gases entering the detection part and to dry the air. The control component receives signals from the temperature sensor and the humidity sensor, as well as the ammonia concentration sensor, and activates the electronic refrigeration plate 22, the gas circulation pump 23, and controls the opening and closing of the solenoid valve 25 to perform cooling, heating, and dehumidifying operations. This keeps the detection part at a constant temperature and humidity and in an environment free of interfering gases, effectively turning the detection part into an independent temperature-controlled chamber that maintains the detection elements at a stable temperature for sample analysis, thereby significantly reducing errors caused by detection.

To facilitate the display of the status of the three-effect air filter, the design is as follows: the triple-effect air filter 19 includes a hollow glass cylindrical shell 19-2 with through holes at both ends, rubber sealing plugs are provided at both ends of the hollow glass cylindrical shell, a card slot 19-3 is defined on the box plate, and circular holes 19-1 are defined on both sides of the card slot; and a slot cover is hinged on the card slot; wherein the circular holes on a same side is coaxially arranged at an end of an air inlet pipe, and the annular sealing plugs are embedded in the circular holes. When the hollow glass cylindrical shell 19-2 is inserted into the card slot, the through holes 19-1 on both sides of the hollow glass cylindrical shell 19-2 are coaxially sealed to the corresponding circular openings at both ends of the card slot, with the inlet pipeline end sealed to the second outlet of the gas circulation pump. This allows the gas in the detection part to flow from the vent of the surrounding plate through the gas circulation pump 23 and the triple-effect air filter 19, returning to the space within the detection part through the circular openings of the card slot, thereby addressing the humidity and nitrogen contamination effects in the detection part's gas.

The hollow glass cylindrical shell is filled with acidic gas adsorption particles (silicone rubber), neutral gas adsorption particles (calcium chloride) and alkaline gas adsorption particles (caustic soda) in sequence. When the adsorbent particles inside the hollow glass cylindrical shell reach saturation and change color, the lid can be opened to replace the three-effect air filter tube of the three-effect air filter.

The controlling component includes a CPU processor, a graphics card and a power supply; a display 8 is also provided in the box cover, and a keyboard 16, a USB (universal serial bus) interface 6 and an exhaust fan 7 are also provided in the controlling part; the graphics card, the power supply, the display, the keyboard, the USB interface and the exhaust fan are all electrically connected to the processor. The keyboard and the USB interface are arranged through the box plate, the exhaust fan dissipates heat for the graphics card and the power supply. A plurality of exhaust holes are arranged on the box plate corresponding to the exhaust fan. The CPU processor receives the data transmitted by each elements of the detecting component for processing to obtain a rapid and accurate determination of the total nitrogen and total phosphorus content in the unknown liquid feces sample, and monitors the detection situation in real time through the display.

Furthermore, the hollow electric rotating platform 11-2 is a precision optical hollow electric rotating platform, which includes a base, a hollow rotating platform mounted on the base, and a digital information driver that drives the rotation of the hollow rotating platform. A serial port relay is also installed on one side of the base of the digital information driver. The digital information driver is a servo stepping motor 11-2-1. This hollow electric rotating platform is connected to the control component via the serial port relay, enabling control and feedback of the servo stepping motor's speed, direction, and travel distance. It can also real-time monitor information such as speed, direction, current distance, current position, and limit switch status.

The integrating sphere 13 can be ISP-REF integrating sphere. The mounting frame includes a limiting slot and a U-shaped slot. The limiting slot is a hollow rectangular structure that passes through from a top to a bottom. The top of the limiting slot is coaxially fixed with the U-shaped slot, and the bottom of the U-shaped slot is bolted to install the hollow electric rotating platform to achieve the coaxial arrangement of the hollow part of the hollow electric rotating platform and the sample port at the top of the integrating sphere. Tops of groove walls on both sides of the U-shaped groove are vertically formed with wing plates outside the groove, the wing plates are flush with the top of the surrounding plate. In addition, reinforcing ribs are also provided on an outer side of the groove wall of the U-shaped groove.

The sample cup embedding frame 11-1 includes an insertion tube and a limit frame. The insertion tube is a hollow tube that passes through from a top to a bottom, the insertion tube is adapted to the hollow part of the hollow electric rotating platform. And the top of the insertion tube is coaxially fixed with the limit frame, the limit frame is formed by two layers of hollow discs coaxially fixed by connecting columns. The hollow part of the hollow disc is a hollow portion, the hollow portion of the hollow part and the hollow part of the insertion tube are coaxial to form a sample cup embedding groove, and the limit frame is located on an upper surface of the box plate.

This device is designed to provide a storage solution for sample cups when not in use and to facilitate easy access to the associated sample cups. A sample cup temporary storage box 18 is installed on one side of the card slot of the three-effect air filter, with the opening of the sample cup temporary storage box 18 fixed to the underside of the box plate. The corresponding position on the box plate is made with an access opening. To protect the sample cups from impact during transport, the sample cup temporary storage box is equipped with cushioning foam, which provides both cushioning and insulation.

This disclosure adopts a design concept of integrated portable device, enabling a single unit to perform functions such as light source generation, spectral collection, sample rotation at a fixed angle, temperature control for the collector, computer data analysis, on-site display on a monitor, and real-time data upload, thus meeting the need for rapid on-site testing.

The spectral collection mechanism, hollow rotating mechanism, and temperature regulating component of this disclosure work together; the hollow rotating mechanism allows for 360° unobstructed assistance in collecting spectral information from to be detected feces samples, maximizing the information gathered from cow dung samples in a given time frame. The temperature regulation component adjusts the temperature of the detection environment during spectral information collection, ensuring the detection unit acts as an independent temperature-controlled chamber, allowing the detection elements to analyze samples at a constant temperature and effectively reducing errors. The gas circulation component performs real-time dehumidification and removal of interfering gases within the detection unit, providing a disturbance-free detection environment to ensure the accuracy of spectral signal detection.

The rapid acquisition of test results from the device relies on importing a data model into the processor. This data model is developed using the device to measure nitrogen and phosphorus contents and scan near-infrared reflectance spectral information from liquid samples such as feces and digestive products collected over many points in a concentrated dairy farm in a specific area. The resulting database for that area allows for testing at any location within the concentrated dairy farm in the future.

The specific usage of this disclosure is as follows: bring the on-site detection to a to be testing site. It needs to be powered on and stabilized for 20-30 minutes before testing begins. Before testing, the controlling component receives signals from temperature sensor, humidity sensor, and ammonia concentration sensor. It then activates the electronic refrigeration sheet, gas circulation pump, and controls the opening and closing of the solenoid valves to cool, heat, and dehumidify the detection part, maintaining a constant temperature, humidity, and interference-free gas environment. Specifically, when the temperature sensor detects a temperature above 20-25° C., the electronic ° C. is activated to cool the detection part. When the temperature falls below 20-25° C., the gas circulation pump is activated, and the solenoid valve on the first connecting pipeline is opened to heat the detection part with warm air generated by the CPU. If the relative humidity detected by the humidity sensor exceeds 50% RH or the ammonia concentration exceeds 0.2 ppm, the gas circulation pump is activated, and the solenoid valve on the first and second circulation pipelines are opened. The gas within the detection part is circulated through the three-effect air filter, and the filtered and dried gas is returned to the detection part, achieving dehumidification and interference removal. This creates an optimal environment for testing.

Samples are collected using sample tubes at the detection point, and then the sample cups are inserted into the sample cup holder. The holder with the sample cups is then coaxially inserted into the hollow part of the electric turntable. The computer controls the servo motor of the electric turntable, which rotates the sample cups at a fixed angle to enable the spectral collection module to gather spectral information from the same sample from multiple angles, with the collected information is saved in real time.

The main technical parameters of the main parts involved in this disclosure are shown in Table 1.

TABLE 1
technical parameters of the main parts

| mechanism/ | | |
|---|---|---|
| component | main parts | technical parameter |
| spectrum | spectrometer | focal length F/4 101 MM; signal |
| collection | | to noise ratio >15000:1 @100 ms |
| mechanism | | integration; integral time 1 ms to |
| | | 120 s; linear calibration >99.8% |
| | integrating sphere | ISP-REF integrating sphere |
| | Integral sphere | Hamamatsu indium gallium |
| | matching element - | arsenide detector, pixel |
| | indium gallium | array: 512 |
| | arsenide detector | |
| | Integral sphere | spectral range: 273 nm to 1704 nm; |
| | matching component - | power consumption: 250 mA at 12 |
| | mercury argon lamp | VDC; aperture: 3 mm connection |
| | standard light source | port: SMA905 |
| | Integral sphere | spectral detection range: 900 |
| | matching element - | to1700 nm 150 L/mm |
| | grating | |
| hollow | hollow electric | transmission ratio: 90:1; drive |
| rotating | rotating platform | mechanism: turbine; resolution: |
| mechanism | | 0.001° = 3.6″ (20 subdivisions); |
| | | eccentricity: 5μ; parallelism: |
| | | 80μ; maximum speed: 50°/sec; |
| | | absolute positioning accuracy: |
| | | 0.01° = 36″ |
| | digital information | Modbus protocol, in conjunction |
| | driver | with PC software, enables control |
| | | and feedback of the speed, |
| | | direction, and travel distance of |
| | | stepping motor, detecting |
| | | information such as speed, |
| | | direction, current distance, |
| | | current position, and limit |
| | | switches |
| temperature | electronic | internal impedance: 4 to 4.39 Ω; |
| regulating | refrigeration sheet | maximum temperature difference: |
| component | | 60° C.; cooling power: 12 w |
| | gas circulation | flow rate: 1.1 L/min; pressure: |
| | pump | 90 kPa; vacuum degree: −40 kPa |
| | temperature sensor | resolution: 0.01° C./0.04% HR; |
| | and humidity sensor | accuracy error: 0.3; |
| | | repeatability: 0.1; |
| | | nonlinear <0.1; |
| | | long term drift <0.04 |
| | ammonia | resolution: 1 ppm |
| | concentration | |
| | sensor | |
| | Hydrogen sulfide | resolution: 0.2 ppm |
| | concentration | |
| | sensor | |
| sample cup | infrared | measurement wavelength: 8 to |
| monitor | temperature sensor | 14 μm; distance coefficient: |
| mechanism | | 20:1; |
| | | response time: 300 ms; |
| | | resolution: 0.1° C. |
| | vibration sensor | measurement range: 0 to 100 um, |
| | | measurement frequency: 5 to |
| | | 1000 Hz |

Compared to the existing laboratory testing, this disclosure does not require acid addition, titration, dilution, filtration, or other preprocessing throughout the entire process. It meets the urgent need for rapid on-site nutrient determination and analysis in dairy farms returning manure to the soil. It addresses technical bottlenecks such as poor timeliness, in low accuracy, and weak stability conventional laboratory chemical determinations, and also solves the challenges of on-site testing affected by the manure treatment environment.

For example, in sampling environment 1, near a certain farm's waste pool, the air relative humidity is 91.2%, and the ammonia concentration is 1.65 ppm.

Table 2 compares the rapid on-site detection results of nitrogen and phosphorus content in manure samples from a mixed waste pool at FY Dairy Farm in Tianjin Binhai New Area. The relative error is calculated as the absolute value of the laboratory reference value minus the measurement value from this device, divided by the laboratory reference value.

TABLE 2
testing items and values

| testing | no | with | 0-15° C., | | | | laboratory |
|---|---|---|---|---|---|---|---|
| items | rotation | rotation | 30-45° C. | 20-25° C. | filtered | filtered | reference |
| TN | 2348-2900 | 2472-2776 | 2290-2958 | 2446-2802 | 2232-3015 | 2420-2828 | 2624 |
| (mg/L) | | | | | | | |
| relative | 10.52 | 5.79 | 12.73 | 6.78 | 14.94 | 7.77 | |
| error | | | | | | | |
| (%) | | | | | | | |
| TP | 430-482 | 444-468 | 410-502 | 435-477 | 390-522 | 426-486 | 456 |
| (mg/L) | | | | | | | |
| relative | 5.7 | 2.63 | 10.09 | 4.61 | 14.47 | 6.58 | |
| error | | | | | | | |
| (%) | | | | | | | |

Although this document discloses specific embodiments and illustrations of the disclosure for explanatory purposes, those skilled in the art will understand that various substitutions, modifications, and changes are possible without departing from the spirit and scope of the disclosure and its accompanying claims. Therefore, the scope of this disclosure is not limited to the content disclosed in the embodiments and illustrations.

What is claimed is:

1. An on-site detection device for nitrogen and phosphorus contents in liquid feces based on near infrared spectroscopy, comprising a box body, a box plate and a box cover, an upper end of the box body is defined with a box opening, the box plate is installed at the box opening, and the box cover is hinged on one side of the box body;
wherein one side of the box body is enclosed by a surrounding plate to form an independent detection part, in which a detecting component and a sensor group are arranged; and another side of the box body is a controlling part, in which a controlling component is arranged; a temperature regulating component and a gas circulation component are also arranged inside the box body; and the detecting component, the temperature regulating component, the gas circulation component and the sensor group are all electrically connected to the controlling component; and
the controlling component in configured to receive an infrared spectrum signal fed back by the detecting component, and analyzes the nitrogen and phosphorus contents of the to be detected liquid feces sample through the characteristics of the infrared spectrum signal; the controlling component is configured to receive a signal value transmitted from the sensor group, and starts the temperature regulating component and the gas circulation component according to the signal value, and the controlling component is also configured to keep the detection environment of the detection part stable to maintain a constant temperature and interference-free detection environment for the detecting component;
wherein the sensor group comprises an ammonia concentration sensor, a temperature sensor and a humidity sensor, and the temperature sensor is configured to monitor a temperature in the detection part and transmit a temperature signal to the controlling component; and the humidity sensor is configured to monitor humidity in the detection part and transmit a humidity signal to the controlling component; and the ammonia concentration sensor is configured to monitor a ammonia content in the detection part and transmit an ammonia content signal to the controlling component.

2. The on-site detection device for nitrogen and phosphorus contents in liquid feces based on near infrared spectroscopy according to claim 1, wherein the detecting component comprises a spectrum collection mechanism and a hollow rotating mechanism, the hollow rotating mechanism is arranged on an upper part of a sample port of an integrating sphere of the spectrum collection mechanism, the hollow rotating mechanism is coaxially embedded with a sample cup embedding frame, a sample cup is coaxially installed in the sample cup embedding frame; and an infrared temperature sensor is installed on the sample cup embedding frame to monitor a detection temperature of the to be detected liquid feces sample in the sample cup in real time, and a vibration sensor is installed on a mounting frame of the hollow rotating mechanism or on an outside of the integrating sphere to monitor in real time and reduce an influence caused by vibrating of the to be detected liquid feces sample on the detection result.

3. The on-site detection device for nitrogen and phosphorus contents in liquid feces based on near infrared spectroscopy according to claim 2, wherein the spectrum acquisition mechanism comprises a spectrometer and the integrating sphere, the integrating sphere is installed on one side of the detection part, the spectrometer is installed on another side of the detection part, and the integrating sphere is communicated with the spectrometer through an optical fiber; the sample port is formed on a top of the integrating sphere; the hollow rotating mechanism comprises the mounting frame, the mounting frame is mounted on a lower surface of the box plate, and a lower end of the mounting frame is coaxially inserted with the integrating sphere, a hollow electric rotating platform is installed in the mounting frame, a middle part of the hollow electric rotating platform is a hollow part, and the sample cup embedding frame is coaxially installed in the hollow part, and the box plate corresponding to the position of the sample cup embedding frame is defined with a through hole, and an upper part of the sample cup embedding frame passes through the through hole.

4. The on-site detection device for nitrogen and phosphorus contents in liquid feces based on near infrared spectroscopy according to claim 1, wherein the temperature regulating component comprises a CPU (central processing unit) processor wrapping box, an electronic refrigeration sheet and a gas circulation pump; wherein the CPU processor wrapping box is configured to collect heat emitted by a CPU processor, the CPU processor wrapping box is provided with a box air outlet; and
a pump air inlet and a pump air outlet of the gas circulation pump are both provided with a three-way valve; and the pump air inlet of the gas circulation pump comprises a first pump air inlet and a second pump air inlet, the pump air outlet of the gas circulation pump comprises a first pump air outlet and a second pump air outlet; wherein the box air outlet of the CPU processor wrapping box is connected to the first pump air inlet of the gas circulation pump through a first pipeline, and the first pump air outlet of the gas circulation pump is connected to the detection part through a second pipeline; and
the electronic refrigeration sheet is arranged on a surrounding plate close to one side of the controlling part, a side A of the electronic refrigeration sheet is a hot side located in the controlling part, and a side B of the electronic refrigeration sheet is a cold side located in the detection part; and
the gas circulation component comprises a three-effect air filter, the gas circulation pump, a first circulation pipeline and a second circulation pipeline; wherein the gas circulation component and the temperature regulating component share the gas circulation pump; and
a plate air outlet is defined on a corner of the surrounding plate, the plate air outlet is connected to the second pump air inlet of the gas circulation pump through the first pipeline, and the second pump air outlet of the gas circulation pump is connected to an air inlet of the triple-effect air filter through the second circulation pipeline, and the air outlet of the triple-effect air filter is connected to the detection part.

5. The on-site detection device for nitrogen and phosphorus contents in liquid feces based on near infrared spectroscopy according to claim 4, wherein the triple-effect air filter comprises a hollow glass cylindrical shell with through holes at both ends, rubber sealing plugs are provided at both ends of the hollow glass cylindrical shell, a card slot is defined on the box plate, and circular holes are defined on both sides of the card slot; and a slot cover is hinged on the card slot; wherein the circular holes on a same side is coaxially arranged at an end of an air inlet pipe, and the annular sealing plugs are embedded in the circular holes.

6. The on-site detection device for nitrogen and phosphorus contents in liquid feces based on near infrared spectroscopy according to claim 5, wherein the hollow glass cylindrical shell is filled with acidic gas adsorption particles, neutral gas adsorption particles and alkaline gas adsorption particles in sequence.

7. The on-site detection device for nitrogen and phosphorus contents in liquid feces based on near infrared spectroscopy according to claim 1, wherein the controlling component comprises a CPU processor, a graphics card and a power supply; a display is also provided in the box cover, and a keyboard, a USB (universal serial bus) interface and an exhaust fan are also provided in the controlling part; the graphics card, the power supply, the display, the keyboard, the USB interface and the exhaust fan are all electrically connected to the processor, and the keyboard and the USB interface are arranged through the box plate, the exhaust fan dissipates heat for the graphics card and the power supply, and a plurality of exhaust holes are arranged on the box plate corresponding to the exhaust fan; the CPU processor receives the data transmitted by each elements of the detecting component for processing to obtain a rapid and accurate determination of the total nitrogen and total phosphorus content in the unknown liquid feces sample, and monitors the detection situation in real time through the display.

8. The on-site detection device for nitrogen and phosphorus contents in liquid feces based on near infrared spectroscopy according to claim 3,
wherein the mounting frame comprises a limiting slot and a U-shaped slot, the limiting slot is a hollow rectangular structure that passes through from a top to a bottom;
wherein a top of the limiting slot is coaxially fixed with the U-shaped slot, and a bottom of the U-shaped slot is bolted to install the hollow electric rotating platform to achieve the coaxial arrangement of the hollow part of the hollow electric rotating platform and the sample port at the top of the integrating sphere;
wherein tops of groove walls on both sides of the U-shaped groove are vertically formed with wing plates outside the groove, the wing plates are flush with the top of the surrounding plate, and reinforcing ribs are also provided on an outer side of the groove wall of the U-shaped groove.

9. The on-site detection device for nitrogen and phosphorus contents in liquid feces based on near infrared spectroscopy according to claim 2, wherein the sample cup embedding frame comprises an insertion tube and a limit frame;
wherein the insertion tube is a hollow tube that passes through from a top to a bottom, the insertion tube is adapted to the hollow part of the hollow electric rotating platform; and the top of the insertion tube is coaxially fixed with the limit frame, the limit frame is formed by two layers of hollow discs coaxially fixed by connecting columns; and the hollow part of the hollow disc is a hollow portion, the hollow portion of the hollow part and the hollow part of the insertion tube are coaxial to form a sample cup embedding groove, and the limit frame is located on an upper surface of the box plate.
